(19) United States
(12) Patent Application Publication
(10) Pub. No.: US 2025/0064383 A1
(43) Pub. Date: Feb. 27, 2025

(54) Systems and Methods for Mapping Wound Features

(71) Applicant: Solventum Intellectual Properties Company, Maplewood, MN (US)

(72) Inventors: Zhijie Zhu, Woodbury, MN (US); Stephen M. Kennedy, St. Paul, MN (US); Sadaf Charkhabi, Cranberry TWP, PA (US); Jeffrey G. Zinn, Roseville, MN (US); Howard M. Kaplan, Mahtomedi, MN (US); Kevin T. Reddy, Minneapolis, MN (US); Shawn C. Dodds, St. Paul, MN (US); Dawn V. Muyres, Stillwater, MN (US); Elias Wilken-Resman, Minneapolis, MN (US); Lindsey L. Hines, Stillwater, MN (US); James D. Carlson, Minneapolis, MN (US); Done Demirgoz, St.Paul, MN (US)

(21) Appl. No.: 18/724,729

(22) PCT Filed: Dec. 13, 2022
(86) PCT No.: PCT/IB2022/062138

§ 371 Date: Jun. 27, 2024

Related U.S. Application Data
(60) Provisional application No. 63/266,192, filed on Dec. 30, 2021.
(60) Provisional application No. 63/407,913, filed on Sep. 19, 2022.

(51) Int. Cl. A61B 5/00; A61B 5/0536

(52) U.S. Cl. CPC A61B 5/445; A61B 5/0536; A61B 5/7475

(57) ABSTRACT

Systems and methods for mapping wound features of a wound bed and also detecting degradation of electrode or connectivity performances are provided. Electrical measurements are collected from an array of electrodes which apply electrical signals to a periwound tissue outside the wound bed. The electrical measurements are processed to generate an impedance map of the wound bed, which is converted to maps conveying spatial distributions of clinical metrics.

### BACKGROUND

Smart wound dressings and other wearable sensors are used to monitor wound healing. The ability to monitor healing may lead to improved healthcare and improved patient outcomes. Monitoring healing may be used to determine whether a current treatment program is effective, or whether changes to a current treatment program should be made.

### SUMMARY

There is a desire to non-invasively measure/monitor (e.g., via mapping and/or quantification) healing for wound beds including open-wound voids and/or subdermal feature voids. In one aspect, the present disclosure provides a method of obtaining wound features of a wound bed. The method includes applying, via an array of sensors (hereinafter exemplified as electrodes), an electrical signal to a periwound tissue outside the wound bed; collecting, via a circuitry functionally connected to the array of electrodes, electrical measurements from the array of electrodes. The method further includes processing, via a processor, the collected electrical measurements to generate one or more impedance maps of the wound bed; and converting the one or more impedance maps to one or more tissue characteristics maps representing a spatial distribution of clinical metrics of the wound bed.

In another aspect, the present disclosure provides a system of obtaining wound features of a wound bed. The system includes an array of electrodes configured to apply an electrical signal to a periwound tissue outside the wound bed, a circuitry functionally connected to the array of electrodes to collect electrical measurements therefrom, and a processor configured to process the collected electrical measurements to generate one or more impedance maps of the wound bed, and convert the one or more impedance maps to one or more tissue characteristics maps representing a spatial distribution of clinical metrics of the wound bed.

In another aspect, the present disclosure provides a device to apply to a wound bed. The device includes an array of electrodes disposed on a periphery of the wound bed and configured to apply an electrical signal to a periwound tissue outside the wound bed, a circuitry functionally connected to the array of electrodes to collect electrical measurements therefrom, and a user interface to receive an instruction from a user, and display information based on the collected electrical measurements. In some cases, the device is a diagnostic or monitoring device. In some cases, the device is a dressing.

### BRIEF DESCRIPTION OF THE DRAWINGS

FIG. 1 is a schematic diagram depicting an example wound measuring system, according to one embodiment.

FIG. 2A is a schematic top view of a tissue site to be tested by a wound measuring system, according to one embodiment.

FIG. 2B is a schematic side view of the tissue site of FIG. 2A.

FIG. 2C is a schematic diagram illustrating a wound measuring system applied to the tissue site of FIGS. 2A-B.

FIG. 3A is a flow diagram of a method of generating a calibration model, according to one embodiment.

FIG. 3B is a plot of change in measured conductivity versus tissue depth in a calibration model, according to one example.

FIG. 4 is a flow diagram of a method of generating a topographical map of wound and would volume estimate, according to one example.

FIG. 5 is a flow diagram of a method of generating wound healing data, according to one example.

FIG. 6 is a line drawing of an 8-electrode device, according to one example.

FIGS. 7A-7C are illustrations of relative conductivity and topographical maps during progression of a wound for Example 1.

FIG. 8A is a graph of wound volume measurements using EIT mapping and comparative methods for Example 3.

FIG. 8B is an illustration of relative conductivity and topographical maps of a wound for Example 3.

FIG. 9A-C show an optical image of an 8-electrode device positioned on a Tissue Mimicking Phantom (TMP) of a wound (A) and EIT maps of the TMP wound model using two different baseline estimation methods (B and C).

FIGS. 10A and 10B show respective unwounded and wounded optical images of a TMP wound model (A1 and B1) and EIT maps of the TMP wound model using a baseline estimation method (A2 and B2).

FIG. 11A-D show optical images of a TMP wound model (A), an 8-electrode device (B), the 8-electrode device positioned on the TMP wound model (C), and an EIT map of the TMP wound model using an fdEIT method (D).

FIG. 12A-C show images of an EIT map of a TMP wound model (A), EIT map of the TMP wound model with a simulated electrode failure (B), and EIT map of the TMP wound model using an algorithm to correct for the simulated electrode failure (C).

In the following description of the illustrated embodiments, reference is made to the accompanying drawings, in which is shown by way of illustration, various embodiments in which the disclosure may be practiced. It is to be understood that the embodiments may be utilized and structural changes may be made without departing from the scope of the present disclosure. The figures are not necessarily to scale. Like numbers used in the figures refer to like components. However, it will be understood that the use of a number to refer to a component in a given figure is not intended to limit the component in another figure labeled with the same number.

### DETAILED DESCRIPTION

As used herein, the term “impedance” refers to an electrical property which a complex quantity including what are referred to as “real” and “imaginary” quantities, e.g., Z=R+iX, where Z is the impedance, R is the so-called real component and is the resistance, and X is the so-called imaginary component and is the reactance. Additionally, the term “conductivity” is used here which is the mathematical inverse of the resistance R. The term “relative conductivity” refers to the conductivity relative to some baseline value, either measured or algorithmically estimated.

The term “electrical measurement(s)” refers to measurement(s) of electrical properties such as conductivity, resistivity, complex impedance, impedance magnitude, capacitance, inductance, admittance, impedance phase angle, reactance, etc., at one or more frequencies.

The term “impedance map(s)” refers to representation(s) of spatial distributions of one or more electrical measurements, which may present in the form of map(s) or other suitable data structures.

The term “tissue characteristics map(s)” or “clinical metrics” refers to representation(s) of spatial distributions of one or more of tissue characteristics including, for example, wound edges/boundaries, wound depth information (e.g., a topographical map of a wound with depth versus x and y coordinates), presence of granulation tissue, granulation tissue thickness, wound healing stage (e.g., hemostasis, inflammation, proliferation, remodeling stage, etc.), epithelial coverage, epithelial layer thickness, biomass, bioburden, infection level, infection type, necrotic tissue, healed tissue, etc. A tissue characteristics map may present in the form of map(s) or other suitable data structures. In some cases, a tissue characteristics representation or map may include a healing metrics.

The term “healing metrics” refers to global assessment(s) of a wound that represent calculations performed on tissue characteristic data. A healing metrics may include wound length, wound width, wound depth (e.g., maximum, minimum, average, etc.), wound area, wound volume, granulation tissue thickness (e.g., maximum, minimum, average, etc.), total epithelial coverage (e.g., percent of a wound bed covered in new epithelium), epithelial thickness (e.g., maximum, minimum, average, etc.), total bioburden, biofilm thickness (e.g., maximum, minimum, average, etc.), biofilm amount, etc.

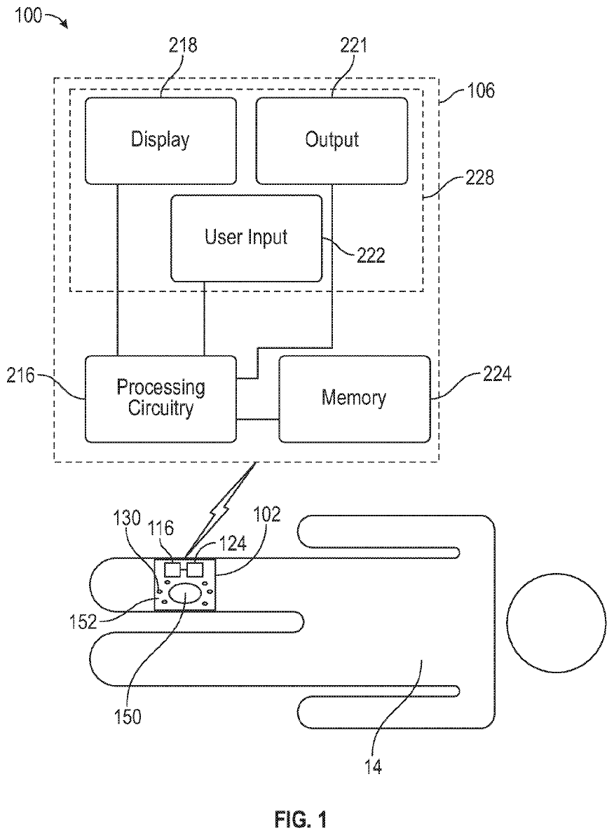

FIG. 1 is a schematic diagram depicting an example wound measuring system 100, according to one embodiment. In the depicted embodiment, the wound measuring system 100 includes a device 102 and a computing device 106. In some cases, the device 102 is a diagnostic or monitoring device. In some cases, the device 102 is a dressing. The device 102 may be communicatively coupled, for example by a wired or a wireless connection, to the computing device 106. The computing device 106 may include processing circuitry 216 coupled to a display 218, an output 221, and a user input 222 of a user interface 228. In some examples, the display 218 may include one or more display devices (e.g., monitor, PDA, mobile phone, tablet computer, any other suitable display device, or any combination thereof). For example, the display 218 may be configured to display physiological information and information indicative of epithelial tissue characteristics determined by wound measuring system 100.

The device 102 may be any type of structure. In some examples, the device 102 may include a bandage including a flexible backing material, an adhesive for bonding to the skin of patient 14, and electrodes 130. In some examples, the device 102 may include a foam dressing including electrodes 130. In some examples, the device 102 may include a material affixed to a tissue via, for example, adhesive, or being physically held in place. In other examples, the device 102 may be a diagnostic patch, for example, a material including any of electrodes 130. In some examples, additional materials may be applied to a patient 14 for wound measuring/monitoring, for example, sterile saline-laden gauze, a gel, or the like, placed between the device 102 and the tissue site 150.

The device 102 includes an array of electrodes 130. When the device 102 is disposed on a wound bed to be tested, the array of electrodes can apply electrical signals from a signal generator to a periwound tissue site 152 outside a tissue site 150. The tissue site 150 may correspond to a wounded tissue or a wound bed, e.g., tissue having damage to epithelial layers and/or subcutaneous tissue. The tissue site 150 may also correspond to tissue having a bruise, tissue having a rash, tissue having an infection, and the like. The tissue site 150 may also correspond to recently healed or currently undamaged tissue that needs to be monitored for the reoccurrence or onset of injury (e.g., to monitor for general tissue health, preservation of healed tissues, viability of reconstructed or donor tissues such as flaps and transplants, edema, Venous Leg Ulcers (VLUs), or Pressure Ulcers (PUs)), in cases where there is not an observable open wound. The periwound tissue site 152 may correspond to tissue in the periwound area which can be defined as the area of skin extending to a certain distance (e.g., several centimeters such as 4 cm) beyond the wound bed, or the surrounding skin extending from the wound bed. In some examples, additional materials may include treatments such as medications and/or may be at least partially electrically conductive and may enhance electrical conductivity between the electrodes 130 and the periwound tissue site 152.

One or more signal generators can be electrically connected to the array of electrodes 130 and configured to generate an alternating electrical signal, e.g., an electrical waveform. The electrical signal may be sinusoidal, a square wave, a pulse wave, a triangle wave, a sawtooth wave, and the like. The signal generator may be configured to generate an electrical signal including one or more frequencies at any frequency, including between 1 kHz to 2 kHz, between 2 kHz to 4 kHz, between 4 kHz to 55 kHz, between 55 kHz to 120 kHz. In some examples, the signal generator may be configured to generate an electrical signal in a frequency range that may be greater than or less than the example ranges above. In some examples, the electrical signal generator may be configured to generate an electrical signal at a predetermined frequency, such as approximately 85 kHz (e.g., 85 kHz #10 kHz). In the example shown, the signal generator is configured to generate electrical signals.

In the depicted embodiment of FIG. 1, the device 102 further includes a processing circuitry 116 and a memory 124. In some embodiments, the device 102 may process the electrical signals without transferring the electrical signals to the computing device 106. For example, the processing circuitry 116 may further include a signal monitor to detect electrical signals applied to the periwound tissue side 152 proximate to the tissue site 150. In other embodiments, the electrical signals, or information corresponding to the electrical signals, may be transferred to computing device 106 for processing, for example, by a wired or wireless connection between the device 102 and the computing device 106.

The memory 116, as well as memory 224, may include any volatile or non-volatile media, such as a random-access memory (RAM), read only memory (ROM), non-volatile RAM (NVRAM), electrically erasable programmable ROM (EEPROM), flash memory, and the like. A memory may be a storage device or other non-transitory medium. A memory may be used by the processing circuitry 216 or 124 to, for example, store fiducial information or initialization information corresponding to physiological monitoring, such as wound monitoring. In some examples, processing circuitry 216 or 124 may store physiological measurements or previously received data from electrical signals in the memory for later retrieval. In some examples, processing circuitry may store determined values, such as information indicative of epithelial tissue characteristics, or any other calculated values, in a memory for later retrieval.

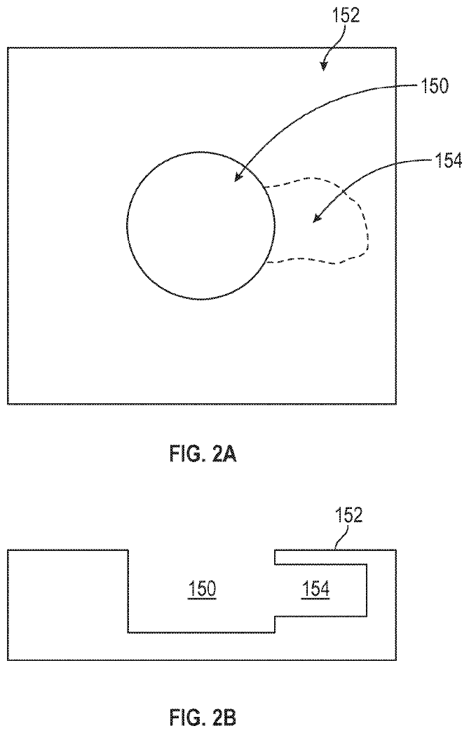

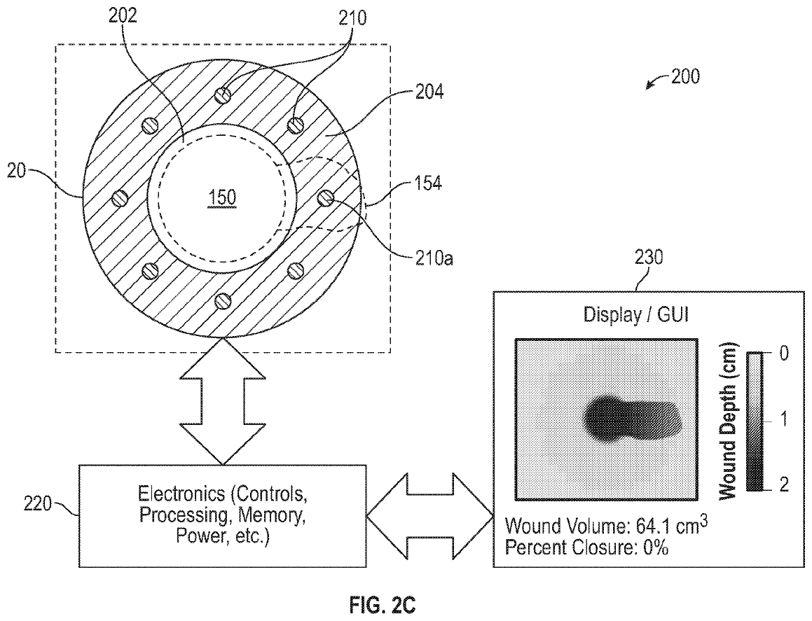

FIG. 2A is a schematic top view of a tissue site to be tested by a wound measuring system 200, according to one embodiment. FIG. 2B is a schematic side view of the tissue site of FIG. 2A. FIG. 2C is a schematic diagram illustrating the wound measuring system 200 applied to the tissue site of FIGS. 2A-B. As shown in the example of FIGS. 2A-B, the tissue site 150 corresponds to an open-wound tissue. The periwound tissue site 152 corresponds to the area surrounding the open-wound tissue 150. A subdermal wound tissue 154 adjacent or connected to the open-wound tissue 150 is beneath the periwound tissue site 152 and thus is unseeable by naked eyes. The total volume of a wound bed includes both the visible open-wound tissue 150 and the volume associated with unseen subdermal features 154 (e.g., tunnels and undermines). It is challenging for conventional clinical methods (e.g., using ruler-based measurement or photo-based measurement) to accurately determine wound volumes because the conventional clinical methods may fail to account for these unseen subdermal volumes which may include a substantial portion of the wound volume at times.

The wound measuring system 200 includes an array of electrodes 210 disposed on the periwound tissue site 152. One or more of the electrodes 210 may be disposed above the subdermal wound tissue 154. In the example shown in FIG. 2C, an electrode 210a is disposed directly above the subdermal wound tissue 154. The array of electrodes 210 is supported by a substrate 20. In the depicted example, the substrate 20 includes a central portion 202 substantially covering the open-wound tissue 150 and a periphery 204 of the central portion 202. The array of electrodes 210 is disposed on an inner surface of the dressing periphery 204.

It is to be understood that in some examples, additional electrodes can be placed on the open-wound. In some examples, electrodes can be exclusively on the periwound tissue, or on both the open-wound and periwound. In some examples, electrodes that reside in the periwound are desirable in that (i) it's less invasive with not having to touch sensitive open-wound tissue and (ii) electrical interface with intact periwound tissue is likely more stable than the open-wound tissue that changes versus time as it heals.

In some examples, electrodes are provided for a 4-proble measurement, with 2 electrodes for current sourcing, and 2 electrodes for voltage measurement), the minimum number of electrodes is 4. In some examples, 8 or more electrodes are provided to obtain mapping results. A higher number of electrodes may enable higher resolution and accuracy.

It is to be understood that any suitable format of electrode devices can be used to enable placement of an electrode array in a wound-circumscribing pattern against periwound tissues. In some examples, the electrode array can be placed on the perimeter of a dressing such as, for example, on the drape of a Negative Pressure Wound Therapy (NPWT) dressing. In some examples, the electrode array can be integrated into a non-dressing diagnostic device. In one example, a flexible circuit substrate can be decorated with snap connectors to which multiple electrodes can be connected. In one example, the device may include a flexible Printed Circuit Board (PCB) with an inner constellation of metal pin electrodes that can interface with the wound bed or with multiple metal pin electrodes that interface with the periwound tissues, circumscribing the wound. Similar configurations can be implemented in a rigid PCB format as well. These electrodes can be placed within the wound bed, outside of the wound bed, or both.

Referring again to FIG. 2C, the system 200 further includes an electronics component 220 electrically connected to the array of electrodes 210. The electronics component 220 may include various control circuitry, processors, memory, power, etc. For example, the electronics component 220 may include one or more of the processing circuitry 116, the memory 124, the processing circuitry 216, and the memory 224 as shown in FIG. 1.

The electronics component 220 is configured to apply, via the array of electrodes 210, electrical signals to the periwound tissue, collect electrical measurements from the array of electrodes 210, process the collected electrical measurements to generate one or more impedance maps of the wound bed (e.g., the open-wound tissue 150, and the subdermal wound tissue 154), and converting the impedance maps to spatial maps of clinical metrics of the wound bed. The obtained wound bed information can be output via a user interface 230, which may include a display, a user input, and an output. As will be articulated later, the one or more impedance maps could also include a baseline map representing unwounded tissue.

Electrical impedance tomography (EIT) is used for measuring and determining spatial distributions of electrical impedance within a continuous two-dimensional (2D) or three-dimensional (3D) space. Typically, impedance measurements are obtained from electrical contacts sparsely distributed in the continuous 2D/3D space, and an impedance map of that continuous 2D/3D is reconstructed by solving the inverse problem of a Finite Element Model (FEM) to space. For wound monitoring applications described herein, an array of electrodes are placed about the perimeter of the wound bed on the periwound tissue in order to spatially map a resistive/conductive profile of the wound and nearby tissues.

The electronics to implement EIT mapping of the wound bed may include the electrodes 210, a microcontroller for measurement of control and data acquisition, a low noise precision current source as a power supply, an Analog-to-Digital (ADC) preamplifier for noise filtering and signal amplification, and input/output multiplexers for switching of electrodes for current sourcing and voltage measurements.

The impedance maps may be obtained at a single frequency or multiple frequencies. In some embodiments, an impedance may be measured with respect to different baselines established through different estimation schemes. The baseline may refer to a map of conductivity values that represent the tissue before wounding. A relative conductivity may refer to the current conductivity map of the tissue subtracted from a baseline map.

In some examples, a baseline measurement of an unwounded tissue may include a homogeneous measurement which captures the background conductivity of the intact tissue, and an inhomogeneous measurement which captures the conductivity of wounded tissue. Methods for estimation of baseline may include, for example, a frequency-difference EIT (fdEIT), measurement-scale features (MSF), best homogeneous (BH) estimators, data-driven estimators, or a combination thereof. Data-driven estimators can include machine learning and deep causal learning methods that use database references. Useful database references can be based on patient history or patient demographics.

As an alternative, it is also possible to reconstruct a conductivity map of wounded tissue using a method that does not require a baseline measurement of unwounded tissue. Because biological tissues can have distinctive frequency responses, impedance distribution of the wound bed can be imaged by a fdEIT reconstruction method, in which measurements are taken under at least two frequencies on wounded tissue. Measurement at the first frequency (i.e., reference frequency) can function as a proxy for a baseline measurement, while measurement at the second frequency (i.e., measuring frequency) serves as an inhomogeneous measurement.

Different methods can be used in reconstructing the conductivity maps. Exemplary methods include a one-step Gauss-Newton (GN) method and an iterative Total Variation (TV) method, in each case applying a suitable hyperparameter. Hyperparameters can be determined heuristically to optimize the degree of contrast between the anomaly and background. Note this heuristic selection can also be replaced with automatic selection based on any pre-determined strategy. The one-step GN method is capable of providing real-time reconstruction results with acceptable quality of shape and size of the anomalies. The TV method, being an iterative method, tends to be computationally slower compared to the GN method, but can also afford a higher resolution of topological features.

As a further alternative, baseline measurement estimation techniques can be applied to compute a homogeneous conductivity distribution of the unwounded tissue. TdEIT can then take the estimated baseline measurement and the inhomogeneous measurement to reconstruct the conductivity map of the wound bed. For instance, either a best homogeneous approximation or pre-defined MSFs can be used to estimate a baseline measurement. In a EIT system with n_{E} electrodes, the baseline measurement U_{baseline} represents a vector of n_{E} (n_{E}-3) voltage measurements under the setting of adjacent stimulation pattern.

In one embodiment, U_{baseline} is estimated by the following steps. First, a finite element model (FEM) that reflects the geometry of the wound bed and electrode arrangement is generated. Second, a simulated baseline measurement U₀ is obtained from the FEM with a homogeneous conductivity distribution, where baseline conductivity σ₀=1. Third, an inhomogeneous measurement Uₘₑₐₛ is acquired from the wound bed. Finally, U₀ is scaled by a ratio parameter u to estimate the baseline measurement. The baseline vector would thus be expressed as:

U_{baseline}=μU₀

When using BH method for baseline estimation, the ratio parameter u can be expressed as

$$μ = {\sum }_{i = 1}^{{n}_{E}} {\sum }_{j = 1}^{{n}_{E} - 3} [ {( {U}_{meas} )}_{i , j} \cdot {( {U}_{0} )}_{i , j} ] / {\sum }_{i = 1}^{{n}_{E}} {\sum }_{j = 1}^{{n}_{E} - 3} {[ {( {U}_{0} )}_{i , j} ]}^{2}$$

When using MSF operators for baseline estimation, the ratio parameter u can be expressed as

$$μ = \frac{f ( {U}_{meas} )}{f ( {U}_{0} )}$$

where f is defined as the feature operator that maps measurements to a feature value. Exemplary MSF operators include an arithmetic mean, range, midrange, electrode-based average range, and electrode-based average midrange. Mathematical representations for each are provided below:
MSF1: Arithmetic mean

$${f}_{mean} ( U ) = \frac{1}{{n}_{E} ( {n}_{E} - 3 )} {\sum }_{i = 1}^{{n}_{E}} {\sum }_{j = 1}^{{n}_{E} - 3} {U}_{i , j}$$

MSF2: Range

$${f}_{range} ( U ) = max ( U ) - min ( U )$$

MSF3: Midrange

$${f}_{midrange} ( U ) = \frac{1}{2} ( max ( U ) - min ( U ) )$$

MSF4: Electrode-based average range

$${f}_{average} ( U ) = \frac{1}{{n}_{E}} {\sum }_{i = 1}^{{n}_{E}} ( max \{ {U}_{{n}_{E} , 1} , {U}_{{n}_{E} , 2} , … , {U}_{{n}_{E} , ( {n}_{E} - 3 )} \} - min \{ {U}_{{n}_{E} , 1} , {U}_{{n}_{E} , 2} , … , {U}_{{n}_{E} , ( {n}_{E} - 3 )} \} )$$

MSF5: Electrode-based average midrange

$${f}_{avemidrange} ( U ) = \frac{1}{2 {n}_{E}} \text{} {\sum }_{i = 1}^{{n}_{E}} ( max \{ {U}_{{n}_{E} , 1} , {U}_{{n}_{E} , 2} , … , {U}_{{n}_{E} , ( {n}_{E} - 3 )} \} + min \{ {U}_{{n}_{E} , 1} , {U}_{{n}_{E} , 2} , … , {U}_{{n}_{E} , ( {n}_{E} - 3 )} \} )$$

In some embodiments, new impedance maps may be arithmetically calculated from impedance maps at given frequencies and from maps generated from different baseline estimation schemes. The measured impedance may vary because of variation in the electrical characteristics from tissue site to tissue site of an individual, e.g., different tissue locations on the same patient and/or animal, variation of tissue characteristics at different times, and variation from individual to individual, e.g., from patient to patient and/or animal to animal. For example, electrical characteristics of tissue may vary based on tissue composition and thickness, tissue water content and/or tissue hydration, ambient relative humidity at the time of measurement, and the like. In addition, electrical characteristics of tissues may depend on what specific tissue types are resident at that location. For example, variations are observed for muscle versus fat-laden tissues, and for the case of wounded tissues, unwounded, well-epithelialized tissues versus open-wound areas with various types and amounts of healed tissues in them (e.g., different amounts of granular tissue filing in the wound bed and/or different degrees of epithelial coverage atop the wound bed).

The obtained impedance maps can be used to determine the wound boundaries and other wound features. In some embodiments, a map of wound tissue impedances can be translated to quantitative metrics of healing. In other words, spatial maps of impedances acquired via EIT (e.g., conductance or resistance) can be translated to quantitative maps of healing such as, for example, wound shape, depth, size (e.g., area or volume), amount of granular tissue, epithelial coverage, wound stage, etc.

In some embodiments, one or more impedance maps can be converted to spatial maps of clinical metrics by calibrating the impedance maps using a calibration model. The clinical metrics can include various wound information data versus (x, y) coordinates in a Cartesian coordinate system (x, y, z) where the z axis corresponds to the depth direction of the wound bed. Example clinical metrics may include wound depth d (x, y), granular tissue thickness t (x, y), epithelial coverage c (x, y), biofilm thickness t (x, y), bioburden (i.e., the number of contaminated organisms found in a given amount of material) b (x, y), infection level i (x, y), healing stage (e.g., inflammation, proliferation, or remodeling stages) indication h (x, y), etc.

In some embodiments, a calibration model can be obtained by correlating an impedance map to physically measured wound data of clinical metrics. In general, the calibration model describes the relationship between the impedance and a clinical metric at various locations (x, y). For example, one calibration model may describe the relationship between an impedance-related property (e.g., conductivity) and the wound depth d (x, y). After such a calibration model is generated by correlating an impedance map to the physically measured clinical metrics, e.g., the wound depths in this case, the calibration model can be used to convert a measured impedance map to a map of wound depth. In similar manner, various calibration models can be generated by correlating an impedance-related property to the corresponding clinical metrics including, for example, wound depth, wound length, wound width, wound area, wound volume, wound topography, granular tissue thickness, epithelial coverage, biofilm thickness, bioburden, infection level, healing stage (e.g., hemostasis, inflammation, proliferation, or remodeling stages), etc. The generated various calibration models can be used to convert a measured impedance map to a map of the corresponding clinical metrics.

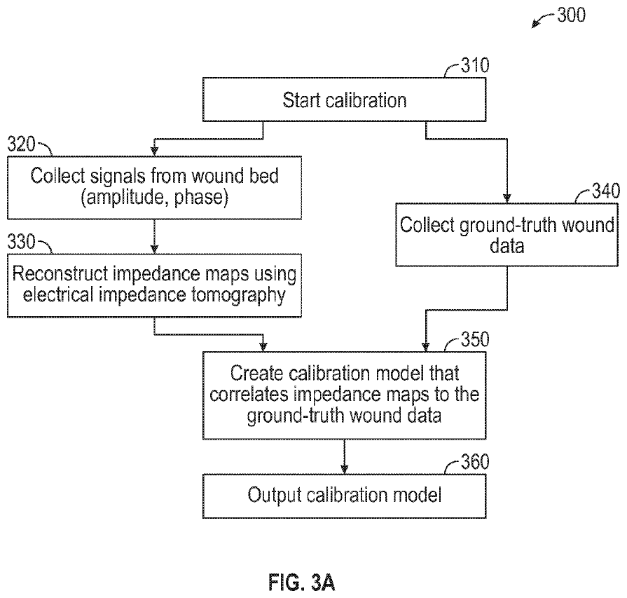

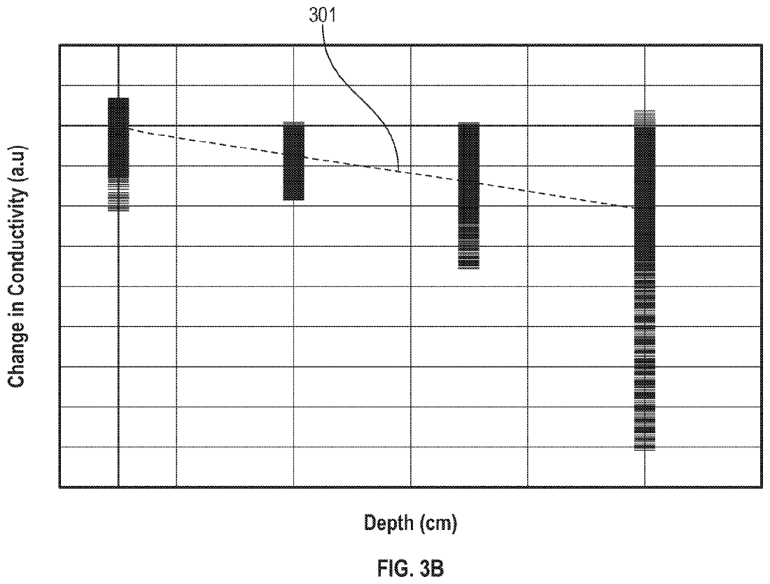

FIG. 3A is a flow diagram of a method 300 of generating a calibration model, according to one embodiment. At 310, calibration wound beds or wound bed models are selected, which may include diverse representations of the wound metric to be calibrated (e.g., for calibrating impedance maps to wound size, wounds or wound models comprising different wound sizes would be needed to generate a strong calibration). At 320, a wound measuring system, such as the system 100 of FIG. 1 and the system 200 of FIG. 2, is used to collect electrical signals from the calibration wound bed. Example electrical signals include voltages (e.g., amplitude, and/or phase) measured from an array of electrodes disposed on a periwound tissue outside a wound bed, such as shown in FIG. 2C. At 330, an impedance map is determined from the spatial distributions of electrical impedance based on the collected electrical signals and under the scheme of electrical impedance tomography (EIT). In one example, the impedance map may be a map of relative conductivity which is reconstructed using the measured voltages and under the scheme of electrical impedance tomography (EIT). At 340, ground-truth data related to one specific type of clinical metrics to be determined such as, for example, wound depth, granular tissue thickness, epithelial coverage, biofilm thickness, bioburden, infection level, healing stage (e.g., inflammation, proliferation, or remodeling stages), etc., can be measured. For example, ground-truth wound depth data for the calibration wound bed can be measured by any suitable means such as, for example, ruler, or camera-based device. At 350, a calibration model is created to correlate the impedance map at 330 and the measured ground-truth data at 340. For example, as shown in FIG. 3B, a calibration curve 301 is created by correlating the change in measured conductivity to the measured tissue depth for the same calibration wound bed. It is to be understood that when the data of impedance-related property and the ground-truth data of clinical metric are obtained for a calibration wound sample, the relationship therebetween can be extracted through various calibration schemes such as, for example, machine learning. At 360, the calibration model is output and can be stored in the electronics component 220 for later retrieval.

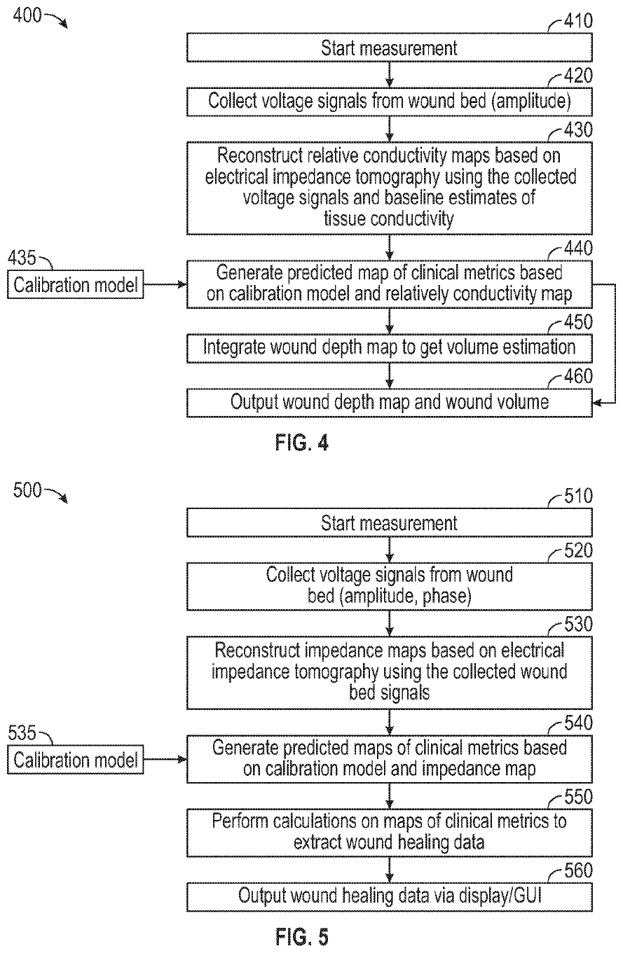

FIG. 4 is a flow diagram of a method 400 of generating a topographical map of wound depth and would volume estimate, according to one example. At 410, the measurement starts by applying, via an array of electrodes, an electrical signal to a periwound tissue at least partially circumscribing the wound bed. At 420, a wound measuring system, such as the system 100 of FIG. 1 and the system 200 of FIG. 2, is used to collect electrical signals from the wound bed. Example electrical signals include an impedance-related property such as voltages (e.g., amplitude, and/or phase) measured from an array of electrodes disposed on a periwound tissue outside a wound bed, such as shown in FIG. 2C. At 430, an impedance map is determined from the spatial distributions of electrical impedance based on the collected electrical signals and under the scheme of electrical impedance tomography (EIT). In one example, the impedance map may be a map of relative conductivity which is reconstructed using the measured voltages and under the scheme of electrical impedance tomography (EIT). At 440, spatial maps for one or more specific types of clinical metrics are generated based on a calibration model from 435 and the impedance map. The specific types of clinical metrics may include, for example, a wound depth, a granular tissue thickness, an epithelial coverage, a biofilm thickness, a bioburden, an infection level, a healing stage (e.g., inflammation, proliferation, or remodeling stages), etc. The calibration model to be used can be obtained by the method of 300 as shown in FIG. 3A. For example, the calibration curve 301 of FIG. 3B can be used to convert the relative conductivity map to a wound depth map. At 450, a wound volume estimation is obtained based on the determined wound depth map by integration of the wound depth values at various sites. At 460, the wound depth map and the wound volume estimation can be output via a user interface such as the user interface 230 of FIG. 2C.

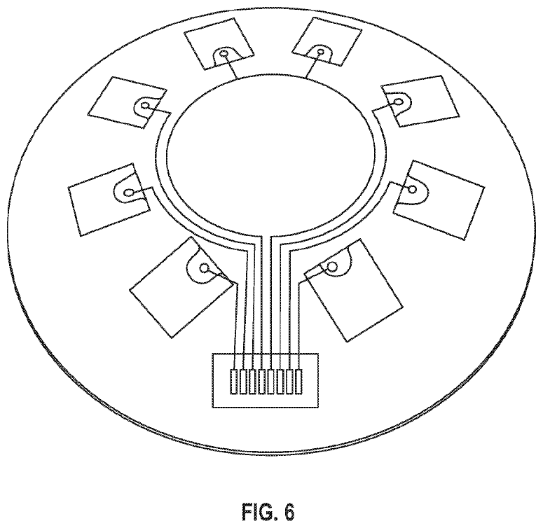

FIG. 5 is a flow diagram of a method 500 of generating wound healing data, according to one example. At 510, the measurement starts by applying, via an array of electrodes, electrical signals to a periwound tissue at least partially circumscribing the wound bed. At 520, a wound measuring system, such as the system 100 of FIG. 1 and the system 200 of FIG. 2, is used to collect electrical signals from the wound bed. Example electrical signals include voltages (e.g., amplitude, and/or phase) measured from an array of electrodes disposed on a periwound tissue outside a wound bed, such as shown in FIG. 2C. At 530, an impedance map is determined from the spatial distributions of electrical impedance based on the collected electrical signals and under the scheme of electrical impedance tomography (EIT). In one example, the impedance map may be a map of relative conductivity which is reconstructed using the measured voltages and under the scheme of electrical impedance tomography (EIT). At 540, maps for one or more specific types of clinical metrics are generated based on a calibration model from 545 and the impedance map. The specific types of clinical metrics may include, for example, a wound depth, a granular tissue thickness, an epithelial coverage, a biofilm thickness, a bioburden, an infection level, a healing stage (e.g., hemostasis, inflammation, proliferation, or remodeling stages), etc. Other healing data other than wound volume that can be calculated based on maps of clinical metrics include, for example, calculation of wound cross-sectional area basted on where the wound's topographical map drop below a certain depth, a total level of infection by spatially integrating the bioburden and/or infection level, a biofilm volume by integrating biofilm thickness, a percent of wound in healing stage X by integrating a map of healing stage and dividing by the total wound area. At 550, wound healing data are extracted by performing calculations on maps of the clinical metrics. Exemplary wound healing data may include, wound volume, wound volume versus time, rate of wound volume closure, wound area, wound area versus time, rate of wound area closure, amount of granular tissue, rate of granular tissue growth, epithelial coverage, rate of epithelialization, infection level, infection risk assessment scores, etc. At 560, the wound healing data can be output via a user interface such as the user interface 230 of FIG. 2C, which can be used as the information indicative of wound information, here a stage of wound healing. Other wound information can include wound depth, granular tissue thickness, epithelial coverage, biofilm thickness, bioburden, and infection level.

Electrodes used in EIT mapping could degrade or fail at any time. Even when all of the electrodes are operating nominally, improper installation or adverse environmental conditions can degrade the adhesive connectivity used to establish electric/ionic conductive path from the tissue surface to the electrode. Degradation in electrode or connectivity performance can produce inaccurate signal readings due to increased contact impedance, thus deteriorating the fidelity of the conductivity map reconstructed with EIT. Advantageously, the principle of voltage-current reciprocity can provide a test to identify these degraded or failed electrodes and to correct the EIT mapping results.

Voltage-current reciprocity provides that data acquired with an excitation and measurement pair of electrodes is equivalent, under ideal conditions, to data obtained by inverting the electrode pairs. In the case of degraded or failed electrodes, the affected measurements will generally not follow the voltage-current reciprocity principle. When the erroneous electrode is part of the excitation pair, less current will flow through the electrode if the electrode contact impedance reaches the maximum external load of the constant current source. When the erroneous electrode is part of the measurement pair, inconsistent data will be acquired since one input of the differential amplifier is floating. In these scenarios, the voltage-current reciprocity principle will be violated.

A reciprocity error for voltage measurement V is obtained by comparing a measured electrical measurement to a predicted electrical measurement based on voltage-current reciprocity. The reciprocity error e can be defined as:

$${e}^{2} = {( V - {V}_{R} )}^{2}$$

where VR denotes the reciprocal measurement of V.

Here, if V is the voltage difference between electrode a and b when sourcing current from electrode c and d, then VR is the voltage difference between electrode c and d when sourcing current from electrode a and b. To properly reconstruct an EIT map with erroneous measurements from degraded or failed electrodes, a weight parameter o based on this reciprocity error can be introduced to the EIT reconstruction algorithm:

$${σ}^{2} = exp ( - \frac{{e}^{2}}{τ} )$$

where t is a unitless constant that can be determined empirically.

Advantageously, the weight parameter makes it possible to algorithmically compensate for variations in electrode performance and/or connectivity performance. With zero reciprocity error, this weight parameter is equal to 1 so that no weighting effect is applied. In this scheme, a large reciprocity error e² corresponds to a value smaller than 1 so that the measurements from the erroneous electrodes are given a less negative effect on the EIT mapping result. To track the degradation state of each electrode, the mean weight value σ of all the measurements corresponding to each electrode can be computed:

$${\overline{σ}}_{i} = \frac{1}{N} {\sum }_{j = 1}^{N} {\overline{σ}}_{i , j}$$

where σᵢ is the mean weight for electrode i, and N is the number of measurements that involve electrode i.

Various embodiments are provided that are dressings, systems of obtaining wound features of a wound bed, and methods of obtaining wound features of a wound bed. It is to be understood that any of embodiments 1-10, 11-13 and 14-15 can be combined. Embodiment 1 is a method of obtaining wound features of a wound bed, the method comprising:
applying, via an array of electrodes, one or more electrical signals to a periwound tissue outside the wound bed;
collecting, via a circuitry functionally connected to the array of electrodes, electrical measurements from the array of electrodes;
processing, via a processor, the collected electrical measurements to generate one or more impedance maps of the wound bed; and
converting the one or more impedance maps to one or more tissue characteristics maps representing a spatial distribution of clinical metrics of the wound bed.

Embodiment 2 is the method of embodiment 1, wherein converting the one or more impedance maps to one or more tissue characteristics maps further comprises obtaining a calibration model by correlating the electrical measurements to physically measured wound data related to the clinical metrics of the wound bed.

Embodiment 3 is the method of embodiment 2, wherein obtaining the calibration model further comprises obtaining a calibration curve by correlating measured relative conductance values to wound depth values.

Embodiment 4 is the method of embodiment 2 or 3, further comprising converting the one or more impedance maps to one or more tissue characteristics maps by using the calibration model.

Embodiment 5 is the method of any one of embodiments 1-4, wherein the one or more impedance maps include a spatial map of conductivity, resistivity, conductance, resistance, reactance, capacitance, inductance, impedance magnitude, impedance phase angle, complex impedance, or a combination thereof at one or more sampling frequencies.

Embodiment 6 is the method of any one of embodiments 1-5, wherein the one or more tissue characteristics maps include a spatial map of wound depth, granular tissue thickness, epithelial coverage, biofilm thickness, bioburden, infection level, or healing stage of the wound bed.

Embodiment 7 is the method of any one of embodiments 1-6, further comprising displaying, via a graphic user interface (GUI), the one or more tissue characteristics maps and system status, and receiving, via the GUI, system configuration parameters from a user.

Embodiment 8 is the method of any one of embodiments 1-7, further comprising determining a volume of the wound bed from the one or more tissue characteristics maps, wherein the wound bed includes one or more subdermal features.

Embodiment 9 is the method of any one of embodiments 1-8, further comprising determining at least one of tissue characteristics including a wound cross-sectional area, a total level of infection, a biofilm volume, a biofilm coverage area, a total volume of granular tissues, an average granular tissue thickness, an average wound depth, a wound length, a wound width, a total epithelial coverage percentage, an average epithelial thickness, or a total epithelial volume based on the one or more tissue characteristics maps.

Embodiment 10 is the method of any one of embodiments 1-9, further comprising outputting information indicative of tissue characteristics based on the one or more tissue characteristics maps.

Embodiment 11 is a system of obtaining wound features of a wound bed, the system comprising:
an array of electrodes configured to apply an electrical signal to a periwound tissue outside the wound bed;
a circuitry functionally connected to the array of electrodes to collect electrical measurements therefrom; and
a processor configured to:
   process the collected electrical measurements to generate one or more impedance maps of the wound bed; and
   convert the one or more impedance maps to one or more tissue characteristics maps representing a spatial distribution of clinical metrics of the wound bed.

Embodiment 12 is the system of embodiment 11, wherein the processor is further configured to determine information indicative of a stage of wound healing based on the one or more tissue characteristics maps.

Embodiment 13 is the system of embodiment 11 or 12, further comprising a graphic user interface (GUI) to display the one or more tissue characteristics maps and system status, and to receive system configuration parameters from a user.

Embodiment 14 is a device to apply to a wound bed comprising:
an array of electrodes disposed on a periphery of the wound bed, and configured to apply an electrical signal to a periwound tissue outside the wound bed;
a circuitry functionally connected to the array of electrodes to collect electrical measurements therefrom; and
a user interface to receive an instruction from a user, and display information based on the collected electrical measurements.

Embodiment 15 is the device of embodiment 14, wherein the circuitry is further configured to:
process the collected electrical measurements to generate one or more impedance maps of the wound bed; and
convert the one or more impedance maps to one or more tissue characteristics maps representing a spatial distribution of clinical metrics of the wound bed.

Embodiment 16 is the method of any one of embodiments 1-10, wherein the one or more impedance maps of the wound bed are algorithmically estimated and do not require a baseline measurement of unwounded tissue, wherein the one or more impedance maps of the wound bed are algorithmically estimated using frequency-difference EIT (fdEIT), measurement-scale features (MSF), best homogeneous (BH) estimators, data-driven estimators, or a combination thereof.

Embodiment 17 is the method of embodiment 16, wherein the one or more impedance maps of the wound bed are algorithmically estimated using measurement-scale features (MSFs) comprising an arithmetic mean, a range, a midrange, an electrode-based average range, an electrode-based average midrange, or a combination thereof.

Embodiment 18 is a method of obtaining wound features of a wound bed, the method comprising:
applying, via an array of electrodes, one or more electrical signals to a periwound tissue outside the wound bed;
collecting, via circuitry functionally connected to the array of electrodes, electrical measurements from the array of electrodes;
quantifying one or more electrode or connectivity performances;
processing, via a processor, the collected electrical measurements to generate one or more impedance maps of the wound bed, wherein the processor algorithmically compensates for degradation of one of more electrode or connectivity performances; and
converting the one or more impedance maps to one or more tissue characteristics maps representing a spatial distribution of clinical metrics of the wound bed.

Embodiment 19 is the method of embodiment 18, wherein the one or more electrode or connectivity performances are detected by performing a test based on voltage-current reciprocity.

Embodiment 20 is the method of embodiment 18 or 19, wherein algorithmically compensating for the degradation of one or more electrode or connectivity performances comprises applying a weight parameter to electrical measurements corresponding to the one or more electrode or connectivity performances.

### EXAMPLES

These examples are merely for illustrative purposes and are not meant to be limiting on the scope of the appended claims.

### Circumferential Electrode Device

An 8-electrode device shown in FIG. 6 was made on a soft and flexible substrate. The electrodes were 3M RED DOT 2360 electrodes (3M Company, St. Paul, MN). The device included a UV-curable silicone encapsulant protective layer, and a urethane film which was laser-etched and bladed with silver to serve as electrical leads and contacts, with the electrodes embedded in the cured silicone encapsulant.

### Electrical Impedance Tomography (EIT) Hardware and Methods

The electronics used to implement EIT mapping of simulated wound environments included a microcontroller for measurement of control and data acquisition, a low noise precision current source as a power supply, an analog-to-digital converter (ADC) preamplifier for noise filtering and signal amplification, and input/output multiplexers for switching of electrodes for current sourcing and voltage measurements. Switching of electrodes for current sourcing and voltage measurement was controlled using CD74HC4067 multiplexers (Texas Instruments, Dallas, TX). Alternating current with a frequency of 20 kHz (amplitude adjustable from 0.1 mA to 2 mA) was supplied to the simulated wound by a Keithley 6221 current source (Keithly Instruments, Solon, OH). The voltage measurements from the electrode pairs were amplified about 17 times using an INA 128 instrumentation amplifier (Texas Instruments) with an appropriate resistor/potentiometer feedback arrangement. To dampen ambient electromagnetic interference such as fluorescent light ballasts (e.g., 50 kHz) and power line noise (e.g., 60 Hz), the amplified signal was filtered by a low pass filter with a cutoff frequency at 31.2 kHz, followed by a high pass filter with a cutoff frequency at 4.8 kHz. The filtered signal was eventually biased by 1.65 V (INA 111AP amplifier, Texas Instruments) and amplified by about 12 times by the INA 128 instrumentation amplifier before it was fed to a 12-bit 3.3V ADC input port of the microcontroller. Electrical Impedance Tomography and Diffuse Optical Tomography Reconstruction Software (EIDORS) v3.9 scripts in MATLAB (Mathworks, Natlick, MA) were used to solve for the EIT reconstruction maps. A one-step Gauss-Newton inverse model was adopted as the inverse solver for reconstruction of conductivity distribution from the measured voltages. The hyperparameter and background value for the inverse model were set manually based on the conductivity of the tissue-mimicking phantom (TMP) models.

### Tissue-Mimicking Phantoms (TMPs) of Wounds Without Simulated Subdermal Features

Tissue mimicking phantom (TMP) wound models were prepared as gelatin-based, proxy tissue structures to simulate the electrical properties of intact skin and granulation tissues of a wound (i.e., dielectric and conductive properties),

Artificial intact skin tissue was prepared as a mixture of water (230 g), gelatin (34.1 g), sodium chloride (1.4 g), vegetable oil (75.0 g), and detergent (40.0 g).

Artificial granulation tissue was prepared as a mixture of water (230 g), gelatin (34.1 g), sodium chloride (1.2 g), vegetable oil (15.0 g), and detergent (40.0 g).

Reagent grade gelatin and sodium chloride (99% purity) were obtained from VWR USA, Radnor, PA. The vegetable oil was CRISCO Pure Vegetable Oil (obtained from B&G Foods, Parsippany-Troy Hills, NJ. The detergent was IVORY Ultra Concentrated Dish Washing Liquid Soap (obtained from the Procter and Gamble Company, Cincinnati, OH). To prepare a given artificial tissue, gelatin was mixed with 100 g of deionized water and the mixture was heated to 80° C. in a water bath. The mixture was then cooled to 35° C. while stirring with a homogenizer. Next, the remaining deionized water, sodium chloride, and detergent were added. When the resulting mixture reached 28° C., the vegetable oil was added with mixing.

All TMP wound models were constructed by first pouring artificial intact skin tissue into 9×9 cm Petri dishes to a depth of 1.2 cm immediately after mixing in the vegetable oil to maintain a pourable temperature of about 28° C. The mixtures were cooled overnight to allow the gelatin to set. The resulting product was used as a TMP of unwounded tissue for baseline measurements and was identified as TMP Model 0. For TMP wound models, a cavity with a depth of 1.2 cm was created in the center of a TMP Model 0 using a cylindrical punch/cutter (6 cm diameter). The base of the simulated wound was then created by pouring additional artificial intact skin tissue into the cavity to a depth of 0.3 cm (leaving a 0.9-cm deep open-ended cavity). The tissue was allowed to set overnight. This construction was identified as TMP Model A and served as a TMP model for an open, non-granulated wound.

The TMP models of a simulated, partially granulated wound were prepared by the following procedure. The cavity of a TMP Model A was filled to a depth of 0.3 cm with artificial granulation tissue and the tissue was allowed to set overnight resulting in a partially granulated TMP wound model identified as TMP Model B.

TMP wound models of a simulated, fully granulated wound were prepared by the following procedure. The cavity of TMP Model A was filled to a depth of 0.6 cm with artificial granulation tissue and the tissue was allowed to set overnight. This model served as a fully granulated TMP wound model identified as TMP Model C and also as the base for preparing additional TMP wound models with varying degrees of epithelialization.

TMP wound models of a simulated, partially epithelialized wound were prepared by the following procedure. A circular counter-mold (4 cm diameter) was placed to cover a continuous section in the center of the granulation tissue surface of TMP Model C, leaving a portion of the peripheral granulation tissue surface exposed. Intact skin tissue mixture was added around the counter-mold to fill the cavity to the surface of the cavity opening (i.e., approximately level with the upper surface of the phantom). After setting, the counter-mold was removed, leaving a partially epithelialized TMP wound model identified as TMP Model D.

TMP wound models of a simulated, fully epithelialized wound were prepared by the following procedure. The remaining cavity of TMP Model C was filled with intact skin tissue mixture to the upper surface of the cavity (i.e., approximately level with the upper surface of the phantom) and the intact cell tissue was allowed to set overnight resulting in a fully epithelialized wound model identified as TMP Model E.

### Tissue-Mimicking Phantom (TMP) of a Wound With Simulated Subdermal Tunnel Features

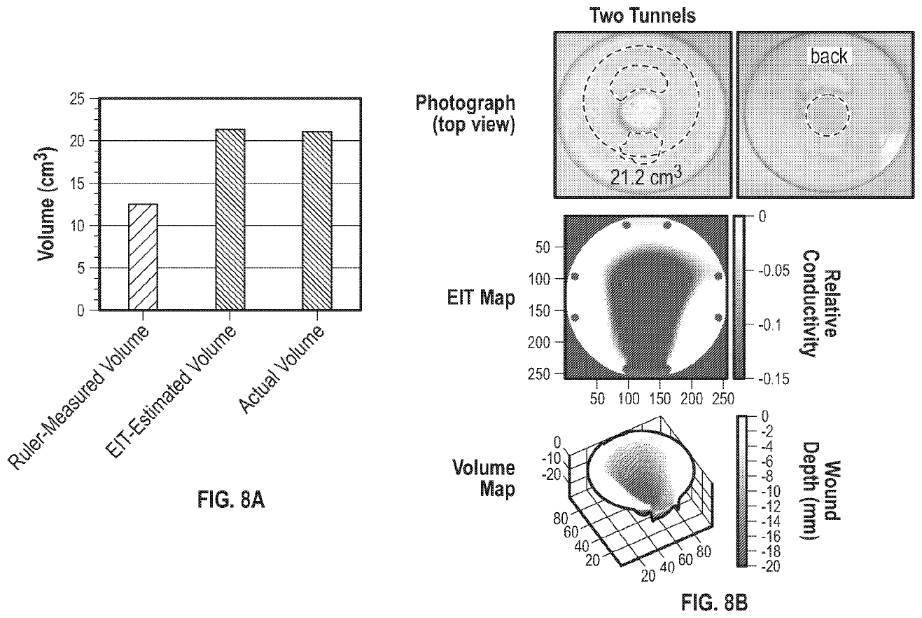

To generate wounds with subdermal features, an ungranulated wound model (TMP Model A) was used. Phantom material was excavated from underneath the periwound area using a spoon-shaped spatula (Bel-Art SP Scienceware Stainless-steel Sampling Spoon and Spatula, ThermoFisher Scientific, Waltham, MA) to generate two simulated subdermal tunnel features that were below the upper surface of the skin phantom and extended from the main cavity as shown in FIG. 8B. These tunnel features increased the total stimulated wound volume by about 70% and were located near and/or at the surface of the Petri dish. This construction was identified as TMP Model F.

### Tissue-Mimicking Phantom (TMP) of an Off-Center Wound Without a Simulated Subdermal Feature (TMP Model G)

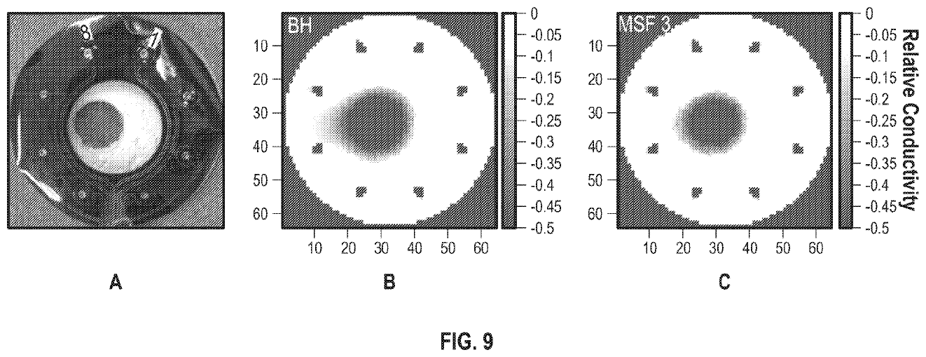

An off-center TMP wound model was constructed by first pouring artificial intact skin tissue into 9×9 cm Petri dishes to a depth of 1.2 cm immediately after mixing in the vegetable oil to maintain a pourable temperature of about 28° C. The mixtures were cooled overnight to allow the gelatin to set. Next, a cavity with a depth of 1.2 cm was created just off the center of each phantom using a cylindrical punch/cutter (6 cm diameter) as shown in photographic image FIG. 9A. The cavity was a hollow channel that extended from the top surface to the bottom surface of the phantom. The gelatin in contact with the dish formed the bottom surface of the phantom and the exposed gelatin surface formed the top surface of the phantom. The cavity served as the simulated wound area. This construction was identified as TMP Model G.

### Tissue-Mimicking Phantom (TMP) of an Off-Center Wound With a Simulated Subdermal Tunnel Feature (TMP Model H)

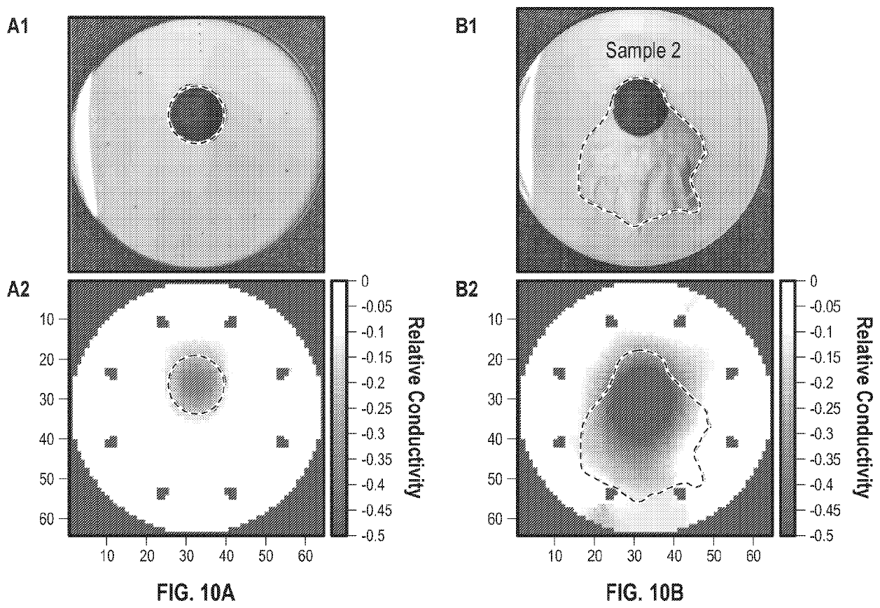

A subdermal tunnel feature was added to TMP Model G (described above) by excavating intact phantom material from the bottom surface of the phantom using a spoon-shaped spatula (Bel-Art SP Scienceware Stainless-steel Sampling Spoon and Spatula). This resulted in a single, large tunnel feature positioned below the upper surface of the phantom that extended from the main cavity into the interior of the phantom. A photographic image of the bottom surface of the finished TMP is shown in FIG. 10C. In this photographic image, dotted lines were superimposed on the image along the outer perimeter of the tunnel region to aid in visualization of the tunnel region. The original cavity plus excavated tunnel region served as the simulated wound area. This construction was identified as TMP Model H.

### EIT Mapping of TMPs

EIT maps were prepared as impedance maps of relative conductivity for TMP Models A-F. The 8-electrode device (described above) was used with a traditional rigid Printed Circuit Board (PCB) and brass pin electrodes. A finite element model was generated based on the location and spacing of the 8 electrodes as well as the shape of the TMP. This electrode device was placed on TMP Models 0 and A-F to acquire and produce spatial maps of the relative conductivity of the wound phantom being measured. In the EIT maps, regions with void spaces had lower measured conductivity that was visually represented by darker coloration (black to gray), while regions filled with artificial tissue had higher measured conductivity that was represented by lighter coloration (light gray to white).

### Example 1. EIT mapping of TMP Models progressing through stages of simulated healing

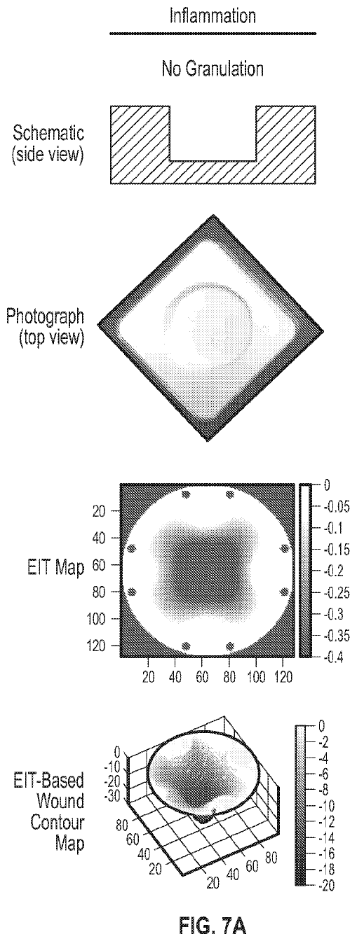

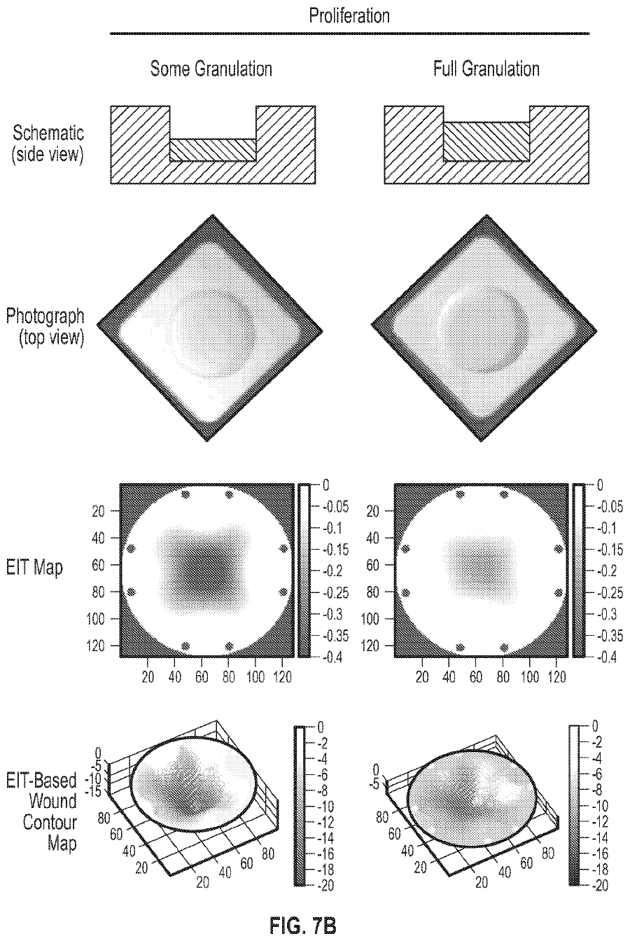

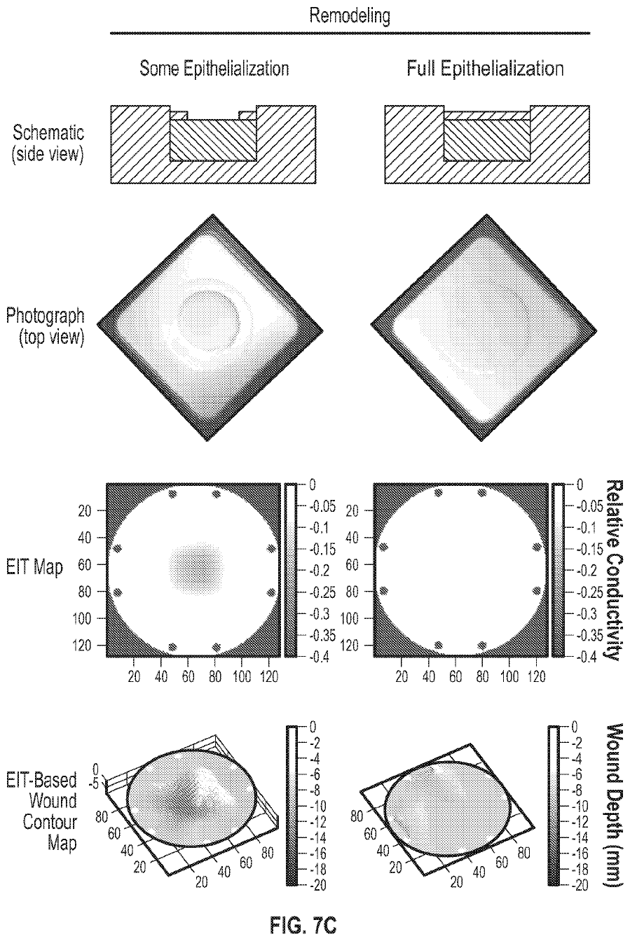

Wound phantoms prepared as TMP Models A-E were used to simulate the progression of wound healing from a relatively fresh wound with no appreciable healing (TMP Model A, no granulation tissue, wound inflammation stage); to TMP Models B and C having partial (some) and full granulation tissue, respectively (wound proliferation stage); and finally to Models D and E having partial (some) and full epithelial coverage, respectively (wound remodeling stage). The method described in the “Electrical Impedance Tomography (EIT) Hardware and Methods” was followed._TMP Model 0 was used for a baseline measurement to construct the EIT maps for TMP Models A-E. The EIT maps (FIGS. 7A-C, ‘EIT Map row’) of TMP Models A-E showed a reduction in dark (black, gray) regions progressing sequentially from the no granulation tissue phantom (TMP Model A) to the full epithelialization phantom (TMP Model E). In FIGS. 7A-C, the EIT map of the no granulation tissue phantom had a large black area representing the cavity, while the map of the full epithelialization map was white to light gray in coloration with no black regions. This demonstrated that EIT maps can be used to visually represent changes in the healing of a wound, including different stages of wound healing.

### Example 2. Estimations of Wound Size and Wound Closure Based on EIT Data

The EIT maps of relative conductivity in FIGS. 7A-C were empirically correlated to physically measured wound depth values. EIT map data from dozens of wound phantoms (having different cavity diameters and depths) were collected to create a calibration model/curve. This yielded 136,488 data points (for each wound phantom, all pixels in each EIT map served as a data point). The data points were plotted on an x/y axis with wound depth about the x-axis and change in conductivity about the y-axis (see, e.g., FIG. 3B). A calibration curve was generated via least-squares linear fitting. The calibration curve was used to correlate the relative conductivity to the wound depth at each site, e.g., by taking a pixel value on the EIT maps and converting that pixel into a depth value,

The calibration curve was directly applied to the EIT maps (in this example, values of relative conductivity versus (x, y) coordinates in the row of “EIT Map” in FIGS. 7-C) to convert them to contour maps of the wounds at different stages of simulated healing (in this example, values of tissue or wound depth versus (x, y) coordinates in the row of “EIT-based wound contour map” of FIGS. 7A-C). These contour plots captured volumetric morphology of the wound phantoms.

Additionally, the EIT-based wound contour maps were used to extract data that clinicians use when monitoring patient wounds. For example, the EIT-based wound contour maps were used to calculate the void volume of the simulated wound by integrating the values of depth versus x- and y-coordinates (Table 1). The ‘true phantom wound volume’ was determined gravimetrically by sequentially: a) weighing the TMP model, filling the void volume of the TMP model with deionized water, c) reweighing the TMP model to determine the added weight, and d) converting the added weight to volume based on the density of water. The EIT calculated volumes of TMP Models A-E closely matched the corresponding measured ‘true phantom wound volumes’

TABLE 1

| | No | Some | Full | Some | Full |
|---|---|---|---|---|---|
| | Granulation | Granulation | Granulation | Epithelization | Epithelization |
| | (TMP | (TMP | (TMP | (TMP | (TMP |
| | Model A) | Model B) | Model C) | Model D) | Model E) |
| EIT-calculated | 39.2 | 21.9 | 11.2 | 6.3 | 1.4 |
| phantom wound | | | | | |
| volume (cm³) | | | | | |
| True phantom | 24.7 | 19.2 | 13.7 | 6.8 | 0 |
| wound volume (cm³) | | | | | |
| EIT-calculated | N/A | 44 | 71.4 | 83.8 | 96.3 |
| phantom wound | | | | | |
| closure percentage | | | | | |
| (%) | | | | | |
| True phantom | N/A | 22.2 | 44.4 | 72.4 | 100 |
| wound closure | | | | | |
| percentage (%) | | | | | |

N/A = not applicable, initial wound volume on which calculations were based

### Example 3. Mapping and Volume Estimation for Wound Phantoms with Tunnel Features

A wound phantom with two excavated subdermal tunnel features (TMP Model F) was mapped using EIT (FIGS. 8A and 8B) as described in Example 1. The locations of the subdermal tunnel features were identified by the EIT map as regions of lower conductivity as indicated by darker shades of coloration (dark gray and black) in the EIT map. In the “volume map” image (FIG. 8B) (calculated using an identical calibration method as described in Example 2), elongated regions of void volume (simulating subdermal wound components) extend from the upper and lower portions of the open section of the phantom in the regions of the tunnels. This topographical wound contour map overlayed very well the physical contours of the total phantom wound space, including both the open wound volumes and covered subdermal volumes.

The void volume of the phantom was determined by three different methods. In method 1, a ruler was used to measure the depth and diameter of the open void and the void volume was calculated based on the measurements and assuming a cylindrical void space (i.e., volume=depth×pi×radius²). This method is often used by clinicians to measure the volume of a wound and has the disadvantage of not measuring the volumes of subdermal features. In method 2, the true volume was measured using water according to the method described in Example 2. The true volume method does include measuring the volumes of subdermal features. In method 3, the EIT based volume calculation was done. The void volume of TMP Model F was determined to be 21.4 cm³ by EIT based mapping, 21.2 cm³ by the true volume measurement method, and 12.6 cm³ by the ruler measurement method.

### Example 4. Mapping of Wound Phantoms using an Algorithmically Calculated Baseline Estimation

The method described in the “Electrical Impedance Tomography (EIT) Hardware and Methods” was followed. An 8-electrode device was prepared from KAPTON polyimide flexible printed circuit board (PCB). The device was equipped with electronic leads and snap-connectors into which 3M RED DOT 2560 electrodes (3M Company) were attached. A finite element model was generated based on the location and spacing of the 8 electrodes as well as the shape of the TMP. The electrode device was placed on the top surface of TMP Model G (FIG. 9A) to acquire and produce spatial maps of the relative conductivity of the wound phantom being measured. A one-step Gauss-Newton method was used to reconstruct the conductivity map, with the hyperparameter set to be 0.3.

Instead of using an actual baseline measurement, BH and MSF3 baseline measurement estimation methods were independently applied to compute a conductivity distribution of the simulated unwounded area. Electrical Impedance Tomography and Diffuse Optical Tomography Reconstruction Software (EIDORS) v3.9 scripts in MATLAB were used for the estimations. The off-center simulated wound locations of lower conductivity were correctly displayed in the reconstructed maps (FIGS. 9A-C). In the individual EIT maps (FIG. 9B using the BH method and FIG. 9C using the MSF3 method), the cavity regions had lower measured conductivity that was visually represented by darker coloration (black to gray), while regions filled with artificial tissue had higher measured conductivity that was represented by lighter coloration (light gray to white). Note that the eight small dark-colored regions on the periphery of the EIT map images resulted from the position placement of the electrodes.

### Example 5. Mapping of Wound Phantoms (With and Without a Subdermal Tunnel Feature) using an Algorithmically Calculated Baseline Estimation

The procedure of Example 4 was followed with the exception that TMP Model G (i.e., TMP model without a simulated subdermal tunnel feature) and TMP Model H (i.e., TMP model with a simulated subdermal tunnel feature) were used with the MSF4 baseline measurement estimation method. The off-center simulated wound locations of lower conductivity were correctly displayed in the reconstructed maps (FIGS. 10B and 10D) In the EIT map for TMP Model G, the cavity region had lower measured conductivity that was visually represented by darker coloration (black to gray), while regions filled with artificial tissue had higher measured conductivity that was represented by lighter coloration (light gray to white). FIG. 10A-image A1 is a photographic image of the top surface of TMP Model G and FIG. 10A-image A2 is the EIT map for TMP Model G.

For TMP Model H, the EIT map displayed a significantly larger region of dark coloration (black to gray) that encompassed both the cavity and subdermal tunnel feature regions (i.e., regions of lower measured conductivity), while regions filled with artificial tissue (i.e., regions of higher measured conductivity) had a lighter coloration (light gray to white). FIG. 10B-image B1 is a photographic image of the bottom surface of TMP Model H and FIG. 10B-image B2 is the EIT map for TMP-H. Note that the eight small dark-colored regions on the periphery of the EIT map images resulted from the position placement of the electrodes. Also note that the dashed lines in FIG. 10A and FIG. 10B were added post-imaging to aid in visualization of the outer perimeter of the cavity and tunnel regions of the TMP models.

### Example 6. Mapping of Wound Phantoms Using an fdEIT Method

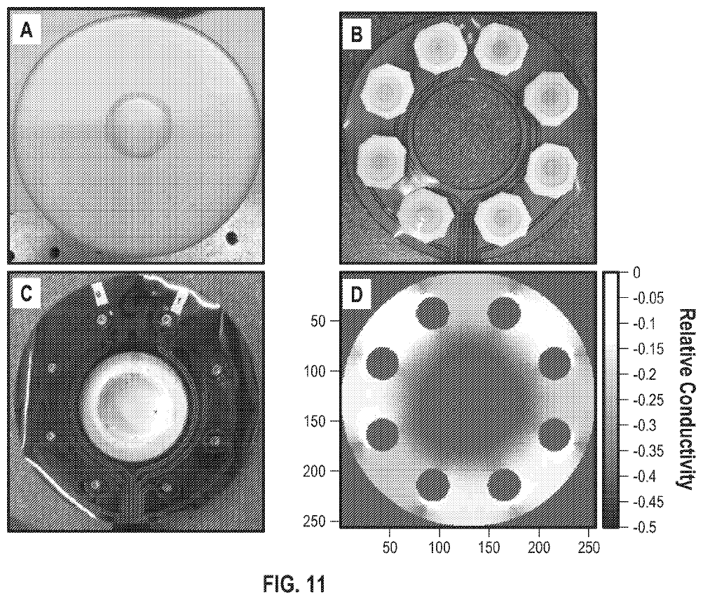

The method described in the “Electrical Impedance Tomography (EIT) Hardware and Methods” was followed using TMP Model A (photographic image FIG. 11A) and the electrode device described in Example 4. The electrode device (photographic image FIG. 11B) was placed on the top surface of TMP Model A (photographic image FIG. 11C) to acquire and produce spatial maps of the relative conductivity of the wound phantom being measured. A one-step Gauss-Newton method was used to reconstruct the conductivity map, with the hyperparameter set to be 0.8. An fdEIT method that utilized two measurements captured with AC current sourcing at frequencies of 2.5 kHz and 40 kHz was used. The homogeneous baseline measurement was captured at 2.5 kHz and the inhomogeneous measurement was captured at 40 kHz. In the reconstructed EIT map (image FIG. 11D), the region of the cavity at the center of TMP Model A had lower measured conductivity that was visually represented by darker coloration (black to gray), while the region of TMP Model A filled with artificial tissue had higher measured conductivity that was represented by lighter coloration (light gray to white).

### Example 7. Detection of Electrode Connectivity Degradation and Algorithmic Compensation of Degraded Electrode Performance by the Application of Electrode Weighting Parameters

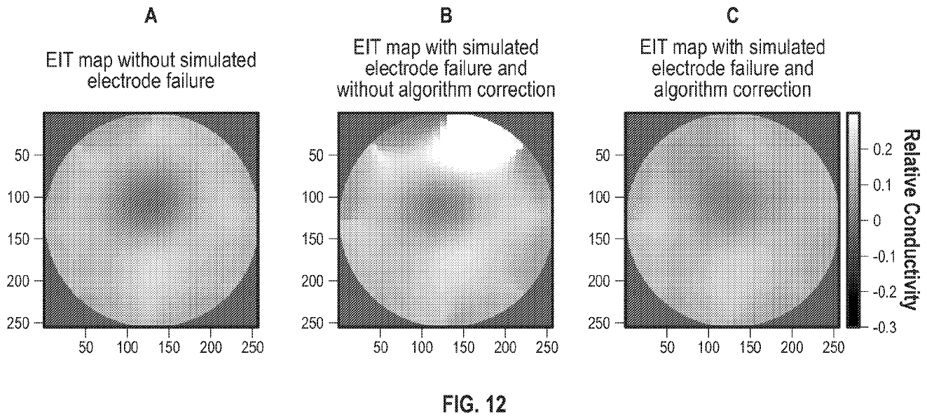

A tank phantom filled with saline solution was used as an artificial proxy structure to simulate the electrical properties and responses of tissue. The tank phantom consisted of a cylindrical plastic tank with an inner diameter of 90 mm, a wall height of 14 mm, and a wall thickness of 2 mm. Eight metallic alligator clips (BU-30 Series, Mueller Electric Co., Akron, OH) were clamped on the vertical wall and evenly distributed along the perimeter of the cylindrical tank. The tank was filled with water until a portion of each alligator clip inside the tank was partially submerged. Salt (NaCl) was then added to create the saline solution that served as the ionic conductive medium. Each alligator clip served as an electrode. A cylindrical non-conductive plastic disc with a diameter of 20 mm and a height of 16 mm was partially submerged in the saline solution at a position slightly off from the center of the tank phantom. The method described in the “Electrical Impedance Tomography (EIT) Hardware and Methods” was used to generate the EIT map of the phantom as a benchmark reference map (image FIG. 12A). The region in the map corresponding to the location of the non-conductive plastic disc was black to dark gray and the remaining region of saline solution surrounding the disc was light gray in color.

The following method was used to simulate erroneous signals from failed electrodes. Forty voltage measurements were taken based on the adjacent stimulation pattern (8 electrode system) to reconstruct a single EIT map. The first five voltage measurements were manually reset to zero voltage and an EIT map from the simulated erroneous signals was generated_(image FIG. 12B). The introduction of erroneous electrode measurements resulted in the appearance of a bright white artifact region located in the upper right perimeter of the EIT map. This artifact region in the EIT map did not faithfully represent the phantom being mapped as the upper right perimeter section of the phantom did not contain high conductivity material. Correction of the EIT map for the simulated electrode failure was conducted by algorithmically calculating reciprocity errors (e²) and applying weight parameters (o) to each electrode measurement as described in the equations and methods above. In the calculations, t (tau) was set at 0.03. The corrected EIT map (image FIG. 12C) was prepared from the weight adjusted electrode measurements. In the corrected map, the artifact region observed in FIG. 12B was removed, while the black to dark gray region corresponding to the plastic disc was maintained.

1. A method of obtaining wound features of a wound bed, the method comprising:
applying, via an array of electrodes, one or more electrical signals to a periwound tissue outside the wound bed;
collecting, via a circuitry functionally connected to the array of electrodes, electrical measurements from the array of electrodes;
processing, via a processor, the collected electrical measurements to generate one or more impedance maps of the wound bed; and
converting the one or more impedance maps to one or more tissue characteristics maps representing a spatial distribution of clinical metrics of the wound bed.

2. The method of claim 1, wherein converting the one or more impedance maps to one or more tissue characteristics maps comprises:
obtaining a calibration model by correlating the electrical measurements to physically measured wound data related to the clinical metrics of the wound bed; and
converting the one or more impedance maps to the one or more tissue characteristics maps using the calibration model.

3. The method of claim 2, wherein obtaining the calibration model comprises obtaining a calibration curve by correlating measured relative conductance values to wound depth values.

4. (canceled)

5. The method of claim 1, wherein the one or more impedance maps comprise a spatial map at one or more sampling frequencies of a measurement selected from the group consisting of conductivity, resistivity, conductance, resistance, reactance, capacitance, inductance, impedance magnitude, impedance phase angle, complex impedance, and a combination thereof.

6. The method of claim 1, wherein the one or more tissue characteristics maps comprise a spatial map of a measurement selected from the group consisting of wound depth, granular tissue thickness, epithelial coverage, biofilm thickness, bioburden, infection level, and healing stage of the wound bed.

7. The method of claim 1, further comprising displaying, via a graphic user interface (GUI), the one or more tissue characteristics maps and system status, and receiving, via the GUI, system configuration parameters from a user.

8. The method of claim 1, further comprising determining a volume of the wound bed from the one or more tissue characteristics maps, wherein the wound bed includes one or more subdermal features.

9. The method of claim 1, further comprising determining at least one tissue characteristic, based on the one or more tissue characteristics maps, wherein the at least one tissue characteristic is selected from the group consisting of a wound cross-sectional area, a total level of infection, a biofilm volume, a biofilm coverage area, a total volume of granular tissues, an average granular tissue thickness, an average wound depth, a wound length, a wound width, a total epithelial coverage percentage, an average epithelial thickness, and a total epithelial volume.

10. The method of claim 1, further comprising outputting information indicative of one or more tissue characteristics based on the one or more tissue characteristics maps.

11. A system of obtaining wound features of a wound bed, the system comprising:
an array of electrodes configured to apply one or more electrical signals to a periwound tissue outside the wound bed;
circuitry functionally connected to the array of electrodes to collect electrical measurements therefrom; and
a processor within or connected to the circuitry and configured to:
process the collected electrical measurements to generate one or more impedance maps of the wound bed; and
convert the one or more impedance maps to one or more tissue characteristics maps representing a spatial distribution of clinical metrics of the wound bed.

12. The system of claim 11, wherein the processor is further configured to determine information indicative of wound information based on the one or more tissue characteristics maps, and wherein the wound information is selected from the group consisting of wound depth, granular tissue thickness, epithelial coverage, biofilm thickness, bioburden, infection level, and healing stage of the wound bed.

13. A device to apply to a wound bed, the device comprising:
an array of electrodes configured to be placed on a periphery of the wound bed, to apply one or more electrical signals to a periwound tissue on the periphery of the wound bed;
circuitry functionally connected to the array of electrodes to collect electrical measurements therefrom; and
a user interface to receive an instruction from a user and display information based on the collected electrical measurements.

14. The device of claim 13, wherein the circuitry is further configured to:
process the collected electrical measurements to generate one or more impedance maps of the wound bed; and
convert the one or more impedance maps to one or more tissue characteristics maps representing a spatial distribution of clinical metrics of the wound bed.

15. The method of claim 1, wherein the one or more impedance maps of the wound bed are algorithmically estimated and do not require a baseline measurement of unwounded tissue, and wherein the one or more impedance maps of the wound bed are algorithmically estimated using a method selected from the group consisting of frequency-difference EIT, measurement-scale features, best homogeneous estimators, data-driven estimators, and a combination thereof.

16. The method of claim 16, wherein the one or more impedance maps of the wound bed are algorithmically estimated using measurement-scale features (MSFs) comprising a value selected from the group consisting of an arithmetic mean, a range, a midrange, an electrode-based average range, an electrode-based average midrange, and a combination thereof.

17. A method of obtaining wound features of a wound bed, the method comprising:
applying, via an array of electrodes, one or more electrical signals to a periwound tissue outside the wound bed;
collecting, via circuitry functionally connected to the array of electrodes, electrical measurements from the array of electrodes;
quantifying one or more electrode or connectivity performances;
processing, via a processor, the collected electrical measurements to generate one or more impedance maps of the wound bed, wherein the processor algorithmically compensates for degradation of the one or more electrode or connectivity performances; and
converting the one or more impedance maps to one or more tissue characteristics maps representing a spatial distribution of clinical metrics of the wound bed.

18. The method of claim 17, wherein the one or more electrode or connectivity performances are detected by performing a test based on voltage-current reciprocity.

19. The method of claim 18, wherein algorithmically compensating for the degradation of the one or more electrode or connectivity performances comprises applying a weight parameter to electrical measurements corresponding to the one or more electrode or connectivity performances.
